(19) United States
(12) Patent Application Publication
(10) Pub. No.: US 2024/0145036 A1
(43) Pub. Date: May 2, 2024

(54) SYSTEM AND METHOD FOR MRNA QUANTIFICATION PROCESSING IN-MEMORY

(71) Applicants: Deliang Fan, Tempe, AZ (US); Fan Zhang, Tempe, AZ (US); Shaahin Angizi, Newark, NJ (US)

(72) Inventors: Deliang Fan, Tempe, AZ (US); Fan Zhang, Tempe, AZ (US); Shaahin Angizi, Newark, NJ (US)

(73) Assignee: Arizona Board of Regents on behalf of Arizona State University, Scottsdale, AZ (US)

(21) Appl. No.: 18/187,203

(22) Filed: Mar. 21, 2023

Related U.S. Application Data
(60) Provisional application No. 63/321,948, filed on Mar. 21, 2022.

(51) Int. Cl. G16B 30/00; G16B 40/00

(52) U.S. Cl. CPC G16B 30/00; G16B 40/00

(57) ABSTRACT

A method of calculating an abundance of an mRNA sequence within a gene comprises storing an index table of the gene in a non-volatile memory, obtaining a short read of the mRNA sequence, generating a set of input fragments from the mRNA sequence, initializing a compatibility table in a volatile memory, for each input fragment in the set of input fragments, searching for an exact match of the input fragment in the index table, calculating a final result from the compatibility table, and calculating an abundance of the mRNA sequence in the gene by aggregating the transcripts compatible with the short read, wherein the calculating step is performed on the same integrated circuit as the non-volatile memory. A system for in-memory calculation of an abundance of an mRNA sequence within a gene is also disclosed.

### CROSS-REFERENCE TO RELATED APPLICATIONS

This application claims priority to U.S. Provisional Application No. 63/321,948, filed on Mar. 21, 2022, incorporated herein by reference in its entirety.

### STATEMENT REGARDING FEDERALLY SPONSORED RESEARCH OR DEVELOPMENT

This invention was made with government support under 2005209 and 2003749 awarded by the National Science Foundation. The government has certain rights in the invention.

### REFERENCE TO A “SEQUENCE LISTING” SUBMITTED AS AN XML FILE

The present application hereby incorporates by reference the entire contents of the XML file named “206339-0021-00US_SequenceListing.XML” in XML format, which was created on Jul. 19, 2023, and is 10,715 bytes in size.

### BACKGROUND OF THE INVENTION

The study of human genetics is a rapidly expanding field, fueled in part by developments in large-scale protein and genomic sequencing technologies. Biopharmaceutical companies and modern healthcare rely heavily on sequencing technologies and the acquired data to develop new drugs and provide effective treatments to patients. However the results obtained from genomic and nucleic acid sequencing present numerous acquisition, production and bioinformatics challenges. The large volume of data obtained in a single sequencing experiment poses a significant logistical challenge to scientists. The resulting quality of data, which often comprises millions to hundreds of millions of small sequence reads, establishes yet another set of challenges as quantifying and verifying these results requires enormous computing power. The development of streamlined, highly automated systems and methods for genomic sequencing and data analysis is critical for transitioning the field from a technology adoption stage to a platform enabling accelerated research and results. Many obstacles are presented in developing methods, algorithms, and computing platforms for the analysis and quantification of sequencing data.

According to the central dogma of molecular biology, a gene contains exons and introns in its structure, where coding exons are translated into proteins. A single gene can encode a set of distinct proteins that participate in diverse biological functions by producing multiple transcripts (i.e., mRNA) with different combinations of exons. To better understand the biological functions and identify important molecular signatures for disease prediction and drug development, efficient and accurate transcript quantification with large-scale mRNA-sequencing (RNA-seq) is critically important (N. L. Bray et al., 2016, Nature biotechnology, vol. 34, no. 5, pp. 525-527; R. Patro et al., 2017, Nature methods, vol. 14, no. 4). High throughput RNA-seq technology is capable of measuring transcript expression by mapping tens of millions mRNA (or DNA) short reads to tens of thousands of annotated genes with each short read containing hundreds of mRNA base pairs (bps). The quantities of various proteins are of great interest as the normalized read coverage on genes or transcripts represents their expression levels.

A typical transcript quantification with RNA-seq requires alignment of short reads to the whole genome or transcriptome before estimating the abundance—a highly time-consuming process. As an example, aligning 30 million short reads from one sample to the reference genome, in the widely used software program TopHat2 (A. Dobin et al., 2013, Biorxiv, p. 000851) takes 28 CPU hours, while quantification of the data with the companion programs (e.g., Cufflinks (C. Trapnell et al., 2010, Nature biotechnology, vol. 28, no. 5, pp. 511-515)) takes another 1-2 CPU hours. Because a read can be mapped to multiple positions, ignoring the full base-to-base alignment of the reads can significantly increase alignment efficiency, and as a result, the quantification efficiency. An alignment-free technique (N. L. Bray et al., 2016, Nature biotechnology, vol. 34, no. 5, pp. 525-527) was developed recently to solve the above issue. The technique focuses on determining the transcripts from which the reads are generated, and not the exact location of the sequence. Without sacrificing overall accuracy, the approach uses k-mer based counting algorithms where each transcript is split into k-length (bps) substrings to enable accurate and efficient mapping with short reads. The novel technique introduced several new bioinformatics tools, e.g., Kallisto (N. L. Bray et al., 2016, Nature biotechnology, vol. 34, no. 5, pp. 525-527) and Salmon (R. Patro et al., 2017, Nature methods, vol. 14, no. 4), which quantifies mRNA abundance without exact position alignment and in a relatively short period of time. However, there is still the intrinsic need in mRNA quantification to map each short read to hundreds of thousands of transcripts, requiring significant computational resources.

Processing-in-Memory (PIM) architecture and logic has gained traction in the last 20 years in solving the memory-wall bottleneck and improving processing time through parallel computing (P. Siegl et al., 2016, MEMSYS '16, p. 295-308; M. Hu et al., 2016, 53rd DAC, ser. DAC '16; K. Kim et al., 2019, ICCAD, pp. 1-8). PIM is considered a promising solution for the “memory-wall” issue in many data-intensive applications, especially within bioinformatics. Existing prior works only explored how to leverage PIM for DNA alignment and DNA assembly (S. Angizi et al., 2020, 57th DAC, pp. 1-6; Z. I. Chowdhury et al., 2020, IEEE JXCDC, vol. 6, no. 1, pp. 80-88; S. Angizi et al., 2019, 56th DAC, pp. 1-6; F. Zokaee et al., 2018, IEEE Computer Architecture Letters, vol. 17, no. 2, pp. 237-240). Recent innovation utilizing PIM for genome alignment and assembly has made great strides in the field, however an important aspect of genome analysis has been overlooked: mRNA quantification. Accurate and efficient mRNA quantification is a crucial step for molecular signature identification, disease outcome prediction as well as drug development. Leveraging PIM logic and architecture to accelerate mRNA quantification is yet to be explored.

Innovation is required to support accurate and efficient quantification of mRNA within bioinformatics and related fields. Novel processing methods and architectures that eliminate frequent data movement between data storage and the computing unit while enabling parallel computing have the potential to save time and energy and accelerate the sequencing process. Thus, there is a need in the art for parallel computing enabled, PIM-based architectures and algorithms to accelerate mRNA quantification allowing for improved processes for molecular signature identification, disease outcome prediction and drug development.

### SUMMARY OF THE INVENTION

In one aspect, a method of calculating an abundance of an mRNA sequence within a gene, comprises storing an index table of the gene in a non-volatile memory, the index comprising a set of nucleotide substrings of length K and having a size in bits of at least 2K, obtaining a short read of the mRNA sequence comprising N nucleotides, generating a set of input fragments of size K from the mRNA sequence using a sliding window, initializing a compatibility table in a volatile memory corresponding to a set of T transcripts of the gene, for each input fragment in the set of input fragments, searching for an exact match of the input fragment in the index table, if an exact match is found, storing a ‘1’ in a position in the compatibility table corresponding to the index of the exact match, calculating a final result having a length T from the compatibility table, wherein each of the T positions of the final result corresponds to one of the set of T transcripts of the gene, and wherein a 1 in the position indicates that the transcript is compatible with the short read, and calculating an abundance of the mRNA sequence in the gene by aggregating the transcripts compatible with the short read, wherein the calculating step is performed on the same integrated circuit as the non-volatile memory.

In one embodiment, the exact match of the input fragment to the index table is calculated with a bitwise XNOR. In one embodiment, the bitwise XNOR is performed in a single clock cycle. In one embodiment, the step of calculating the final result comprises the step of performing a bitwise AND operation between a first set of bits in the compatibility table and a second set of bits in the compatibility table, and storing the result in the compatibility table. In one embodiment, the method further comprises detecting whether all the bits in a subset of the compatibility table are set to 0. In one embodiment, the input fragments are generated using at least one shift register.

In one embodiment, the method further comprises storing the index table and the compatibility table in the same bank. In one embodiment, the method further comprises the step of splitting the index table into multiple index sub-tables stored in different areas of the memory. In one embodiment, the index table is split based on the first nucleotide in the input fragments. In one embodiment, the method further comprises recording an index of the multiple index sub-tables in a look-up table. In one embodiment, the method further comprises querying the look-up table for the correct index sub-table before searching for an exact match of the input fragment in the index table.

In one aspect, a system for in-memory calculation of an abundance of an mRNA sequence within a gene comprises a non-volatile computer-readable memory storing a set of binary values, the non-volatile computer-readable memory comprising a plurality of read bitlines and read wordlines, a computational array communicatively connected to the non-volatile computer-readable memory, comprising an input shift register configured to generate binary substrings from an input binary string, a multiplexer having at least two inputs, having at least one output electrically connected via a resistor to at least one read bitline, configured to selectively change the voltage of the read bitline at the input to a sense amplifier during a read operation, and a set of combinatorial logic gates electrically connected to an output of the sense amplifier, configured to return a result and store the result in the non-volatile computer-readable memory, and a processor configured to calculate the abundance of an mRNA sequence within a gene by storing an index table of the gene in the non-volatile memory, generating a set of input fragments from the mRNA sequence, searching for exact matches of the input fragments in the index table using the multiplexer and the set of combinatorial logic gates to calculate a set of transcripts compatible with the short read, and calculating the abundance of the mRNA sequence in the gene by aggregating the transcripts compatible with the input binary string.

In one embodiment, the set of combinatorial logic gates comprises at least one XNOR gate. In one embodiment, the set of combinatorial logic gates comprises an XNOR gate with one inverted input. In one embodiment, the multiplexer has exactly two inputs and one output. In one embodiment, the computational array comprises first and second sense amplifiers configured to have different threshold voltages. In one embodiment, the system further comprises an all-zero detection unit configured to detect when a calculated result vector contains all zeros. In one embodiment, the computational array and the volatile computer-readable memory are positioned in a single integrated circuit. In one embodiment, the processor is positioned in the single integrated circuit. In one embodiment, the processor is further configured to divide the index table into multiple index sub-tables stored in different areas of the memory.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 is a diagram of a computing device.

FIG. 2 shows the pseudo code of a proposed parallel bit-wise in-memory algorithm for mRNA quantification.

FIG. 3A depicts steps for a method of mRNA quantification in-memory.

FIG. 3B depicts a diagram of an alignment-free method for mRNA quantification.

FIG. 4 depicts the architecture diagram for a Processing-In-Memory (PIM) mRNA quantification, SOT-RAM based computational array containing both k-mer and k-comp array and the peripheral circuitry for the PIM-Quantifier

FIG. 5 is a diagram of an exemplary embodiment for the mRNA quantification-in-memory process and data mapping.

FIG. 6 depicts an exemplary parallel search & matching operation using XNOR logic.

FIG. 7 is a method of calculating an abundance of an mRNA sequence within a gene.

FIG. 8 is a diagram of transient simulation wave-forms of the disclosed quantifier's sub-array and the reconfigurable Sense Amplifier (SA) for performing single-cycle in-memory operations.

FIG. 9 is a diagram of a Monte-Carlo simulation of Vₛₑₙₛₑ for a one-row activation PIM scheme and a conventional two-row activation PIM scheme.

FIG. 10 is a set of graphs of area, power, and throughput of different PIM accelerators and CPU, the normalized throughput of different PIM accelerators and CPU, and the normalized throughput/watt of different PIM accelerators and CPU.

FIG. 11 is a diagram of normalized throughput/power/area of various PIM platforms.

### DETAILED DESCRIPTION

### Definitions

It is to be understood that the figures and descriptions of the present invention have been simplified to illustrate elements that are relevant for a clear understanding of the present invention, while eliminating, for the purpose of clarity, many other elements found in related systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20%, ±10%, ±5%, +1%, and +0.1% from the specified value, as such variations are appropriate.

The term “RNA-Seq” as used herein refers to RNA sequencing, which is a sequencing technique which uses next-generation sequencing (NGS) to reveal the presence and quantity of RNA in a biological sample at a given moment, analyzing the continuously changing cellular transcriptome.

The term MRAM refers to Magnetoresistive random-access memory, which is a type of non-volatile random-access memory which stores data in magnetic domains.

The term “SOT-MRAM” refers to Spin-orbit torque magnetic random-access memory, which are devices featuring switching of the free magnetic layer done by injecting an in-plane current in an adjacent SOT layer, unlike STT-MRAM where the current is injected perpendicularly into the magnetic tunnel junction and the read and write operation is performed through the same path.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, MATLAB, Python, PUP, Perl, Ruby, or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

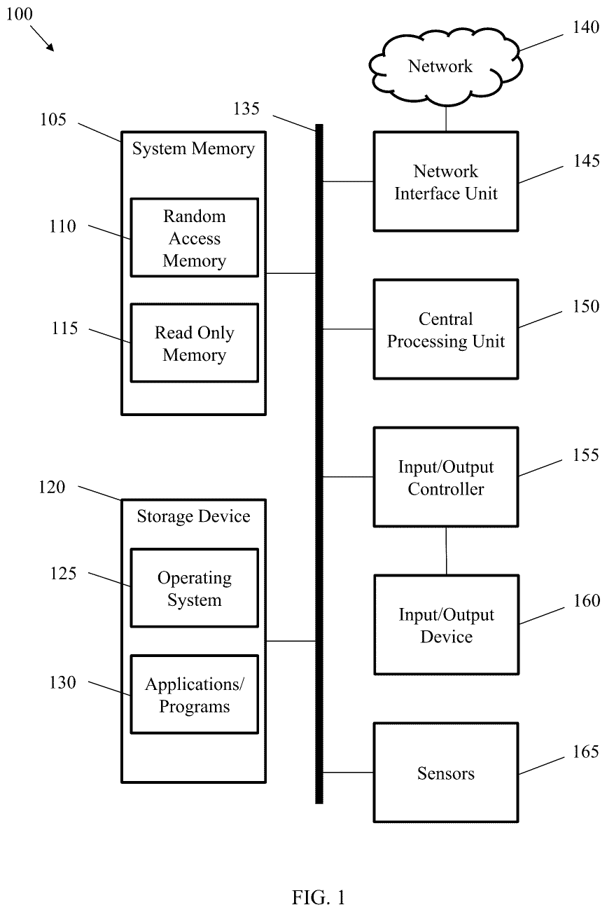

FIG. 1 and the following discussion are intended to provide a brief, general description of a suitable computing environment in which the invention may be implemented. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 1 depicts an illustrative computer architecture for a computer 100 for practicing the various embodiments of the invention. The computer architecture shown in FIG. 1 illustrates a conventional personal computer, including a central processing unit 150 (“CPU”), a system memory 105, including a random access memory 110 (“RAM”) and a read-only memory (“ROM”) 115, and a system bus 135 that couples the system memory 105 to the CPU 150. A basic input/output system containing the basic routines that help to transfer information between elements within the computer, such as during startup, is stored in the ROM 115. The computer 100 further includes a storage device 120 for storing an operating system 125, application/program 130, and data.

The storage device 120 is connected to the CPU 150 through a storage controller (not shown) connected to the bus 135. The storage device 120 and its associated computer-readable media provide non-volatile storage for the computer 100. Although the description of computer-readable media contained herein refers to a storage device, such as a hard disk or CD-ROM drive, it should be appreciated by those skilled in the art that computer-readable media can be any available media that can be accessed by the computer 100.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 100 may operate in a networked environment using logical connections to remote computers through a network 140, such as TCP/IP network such as the Internet or an intranet. The computer 100 may connect to the network 140 through a network interface unit 145 connected to the bus 135. It should be appreciated that the network interface unit 145 may also be utilized to connect to other types of networks and remote computer systems.

The computer 100 may also include an input/output controller 155 for receiving and processing input from a number of input/output devices 160, including a keyboard, a mouse, a touchscreen, a camera, a microphone, a controller, a joystick, or other type of input device. Similarly, the input/output controller 155 may provide output to a display screen, a printer, a speaker, or other type of output device. The computer 100 can connect to the input/output device 160 via a wired connection including, but not limited to, fiber optic, Ethernet, or copper wire or wireless means including, but not limited to, Wi-Fi, Bluetooth, Near-Field Communication (NFC), infrared, or other suitable wired or wireless connections.

As mentioned briefly above, a number of program modules and data files may be stored in the storage device 120 and/or RAM 110 of the computer 100, including an operating system 125 suitable for controlling the operation of a networked computer. The storage device 120 and RAM 110 may also store one or more applications/programs 130. In particular, the storage device 120 and RAM 110 may store an application/program 130 for providing a variety of functionalities to a user. For instance, the application/program 130 may comprise many types of programs such as a word processing application, a spreadsheet application, a desktop publishing application, a database application, a gaming application, internet browsing application, electronic mail application, messaging application, and the like. According to an embodiment of the present invention, the application/program 130 comprises a multiple functionality software application for providing word processing functionality, slide presentation functionality, spreadsheet functionality, database functionality and the like.

The computer 100 in some embodiments can include a variety of sensors 165 for monitoring the environment surrounding and the environment internal to the computer 100. These sensors 165 can include a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor.

Certain embodiments may include In-DRAM Computing which is defined herein as computation or computing that takes advantage of extreme data parallelism in Dynamic Random Access Memory (DRAM). In some embodiments, a processing unit performing In-DRAM computing as contemplated herein may be located in the same integrated circuit (IC) as a DRAM IC, or may in other embodiments be located in a different integrated circuit, but on the same daughterboard or dual in-line memory module (DIMM) as one or more DRAM IC, and may thus have more efficient access to data stored in one or more DRAM ICs on the DIMM. It is understood that although certain embodiments of systems disclosed herein may be presented as examples in specific implementations, for example using specific DRAM ICs or architectures, these examples are not meant to be limiting, and the systems and methods disclosed herein may be adapted to other DRAM architectures, including but not limited to Embedded DRAM (eDRAM), High Bandwidth Memory (HBM), or dual-ported video RAM. The systems and methods may also be implemented in non-volatile memory based crossbar structures, including but not limited to Resistive Random-Access Memory (ReRAM), Memristor, Magnetoresistive Random-Access Memory (MRAM), Phase-Change Memory (PCM), Ferroelectric RAM (FeRAM), Spin-Orbit Torque Magnetic Random Access Memory (SOT-MRAM) or Flash memory.

The system may also include in-memory computation (IMC) (or in-memory computing) which is the technique of running computer calculations entirely in computer memory (e.g., in RAM). In some embodiments, in-memory computation is implemented by modifying the memory peripheral circuitry, for example by leveraging a charge sharing or charge/current/resistance accumulation scheme by one or more of the following methods: modifying the sense amplifier and/or decoder, replacing the sense amplifier with an analog-to-digital converter (ADC), adding logic gates after the sense amplifier, or using a different DRAM cell design. In some embodiments, additional instructions are available for special-purpose IMC ICs.

The system may also include processing in memory (PIM, sometimes called processor in memory) which is the integration of a processor with RAM (random access memory) on a single IC. The result is sometimes known as a PIM chip or PIM IC.

The present disclosure includes apparatuses and methods for logic/memory devices. In one example embodiment, execution of logical operations is performed on one or more memory components and a logical component of a logic/memory device.

An example apparatus comprises a plurality of memory components adjacent to and coupled to one another. A logic component may in some embodiments be coupled to the plurality of memory components. At least one memory component comprises a partitioned portion having an array of memory cells and sensing circuitry coupled to the array. The sensing circuitry may include a sense amplifier and a compute component configured to perform operations. Peripheral circuitry may be coupled to the array and sensing circuitry to control operations for the sensing circuitry. The logic component may in some embodiments comprise control logic coupled to the peripheral circuitry. The control logic may be configured to execute instructions to perform operations with the sensing circuitry.

The logic component may comprise logic that is partitioned among a number of separate logic/memory devices (also referred to as “partitioned logic”) and which may be coupled to peripheral circuitry for a given logic/memory device. The partitioned logic on a logic component may include control logic that is configured to execute instructions configured for example to cause operations to be performed on one or more memory components. At least one memory component may include a portion having sensing circuitry associated with an array of memory cells. The array may be a dynamic random access memory (DRAM) array and the operations can include any logical operators in any combination, including but not limited to AND, OR, NOR, NOT, NAND, XOR and/or XNOR boolean operations.

In some embodiments, a logic/memory device allows input/output (I/O) channel and processing in memory (PIM) control over a bank or set of banks allowing logic to be partitioned to perform logical operations between a memory (e.g., dynamic random access memory (DRAM)) component and a logic component.

Through silicon vias (TSVs) may allow for additional signaling between a logic layer and a DRAM layer. Through silicon vias (TSVs) as the term is used herein is intended to include vias which are formed entirely through or partially through silicon and/or other single, composite and/or doped substrate materials other than silicon. Embodiments are not so limited. With enhanced signaling, a PIM operation may be partitioned between components, which may further facilitate integration with a logic component's processing resources, e.g., an embedded reduced instruction set computer (RISC) type processing resource and/or memory controller in a logic component.

Some embodiments of the systems and methods disclosed herein aim to develop a fast and efficient hardware and software accelerator for the compute- and data-intensive alignment-free mRNA quantification process. The quantification of mRNA is a crucial step in molecular signature identification, disease progression prediction and drug development.

The disclosure is summarized as follows: (1) a PIM-friendly mRNA quantification algorithm, which converts the complex graph processing-based algorithm into primary bulk bit-wise logic operations supported by most PIM architectures; (2) a PIM-Quantifier architecture and circuit, based on emerging non-volatile Spin-Orbit Torque Magnetic Random-Access Memory (SOT-MRAM), optimized for the proposed mRNA quantification algorithm with fast and efficient one-cycle parallel XNOR&AND logic operations; (3) a large gene data partition and mapping algorithm to efficiently deploy the proposed mRNA quantification algorithm into the associated PIM-Quantifier hardware platform, having the potential to enable data parallelism and increase throughput. Displayed in the experimental results is a comparison of the PIM-Quantifier with other recent non-volatile PIM platforms and software implementations (i.e. CPU) paneling both performance and energy efficiency.

mRNA Quantification-In-Memory Algorithm

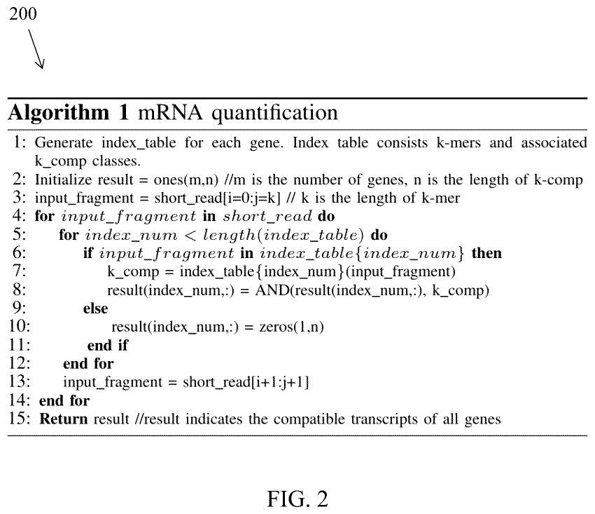

Referring now to FIG. 2, pseudo-code of a novel, alignment free, in-memory mRNA quantification algorithm is described. The algorithm supports a primary bit-wise logic operation of bulk data stored in dynamic random-access memory (DRAM) as alphabetic representations of amino acids contained in mRNA transcription sequences.

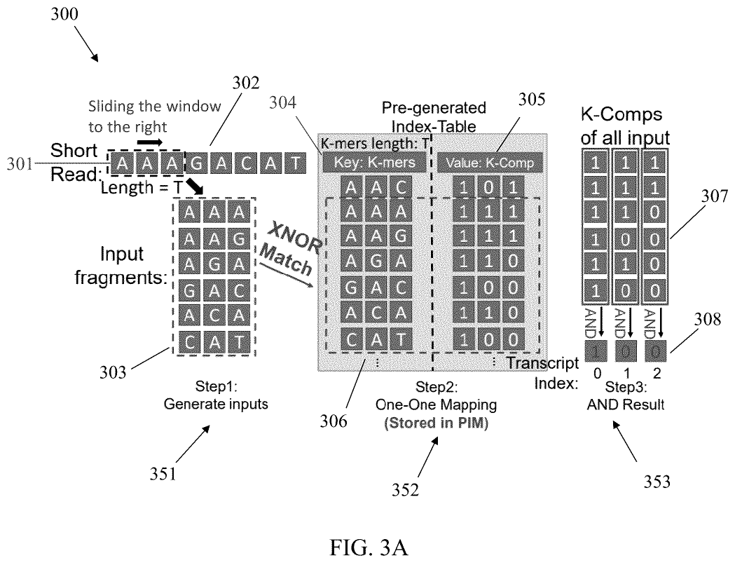

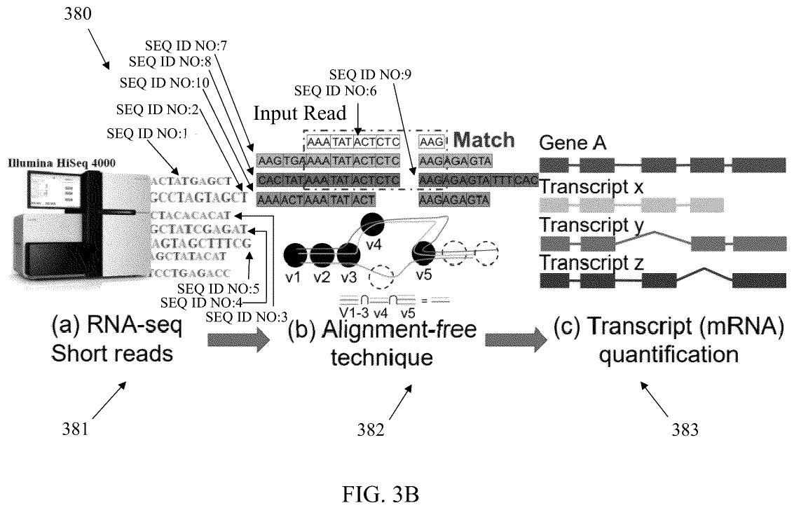

An mRNA quantification-in-memory algorithm is shown in FIG. 2, with further detail shown in FIG. 3A, and includes the following steps. First, each gene is transferred to an index-table which contains two parts: k-mers and k-comp (line-1 in FIG. 2). All transcripts' sequences of a gene are fragmented into k-length substrings, defined as ‘k-mer’, starting from each position; and for each k-mer, the k-compatibility (‘k-comp’) classes are defined according to its presence in the transcript (‘1’ means present’, whereas ‘0’ means ‘not present’). The k-comp classes are represented as a one-dimensional vector with ‘0’s and ‘1’s with a size defined by the number of transcripts in that specific gene. The k-mers along with its k-comp classes are then pushed into a Hashmap. The index-table is then constructed from the k-mers and their k-comp classes. The size of index-table depends on the value of k, and its size can be at most L−k+1, for a gene with length L. Typically, a human genome contains thousands of genes, each gene represented by one index-table which has thousands of k-mers on average, with a k-mer length of 40. The initial index-table construction step is a one-time effort for every gene, and in some embodiments the index-table is pre-generated and stored in the PIM platform.

With reference to FIG. 2, second, a sliding window with the same length as k-mers is used to generate input fragments for every short read (line-13 in FIG. 2). Each fragment will be sent to every gene index-table to search if an exact match (implemented using bit-wise XNOR logic in hardware) will be identified. Once the exact match in one index-table is found, the corresponding k-comp value will be recorded for the next step (line-7 in FIG. 2). If there is no match, it means this short read doesn't belong to any transcript in this gene. Thus, the whole short read should be discarded for this index-table (line-10 in FIG. 2). When all fragments are processed, the corresponding k-comps 305 are collected to conduct bit-wise AND operations (line-8 in FIG. 2). The value-‘1’ and its position in AND logic outputs indicate the corresponding transcript is compatible with current input short read.

To better explain the process, one example is shown in FIG. 3A. The input fragment 303 is generated by the sliding window 301 from the input short read 302, where the input short read is coming from the patient. Each gene generates an index-table 304 with the parameter “k-mer length: T” in the pre-computing stage. In the depicted example, the k-mer length is 3. It is also assumed this gene has 3 transcripts, resulting in the length of k-comp 305 is 3. For example, the k-mer “AAC” has the corresponding k-comp “101”, which means it is occurred in the transcript-0 and transcript-2 in this gene, but not in transcript-1. The K-mer is generated from the reference gene during the pre-processing stage. Again, this index-table only belongs to one gene, which is generated only once in advance and will be continuously used for processing new incoming input short reads. As an example in FIG. 3A, if the input short read 302 length is 8, 6 k-mers with k=3 will be generated. To identify if a short read exists in a transcript, in some embodiments we must compare all the input fragments with the K-mer table. Each fragment will be fed into the pre-generated index-table to find the exact match and its corresponding k-comp. A bit-wise AND operation is then performed on all the selected k-comps 305 to produce the final output 308. In this example, based on the final AND output-‘100’, the transcript-0 is the found compatible transcript. Of course, the final output may have multiple ‘1’s, indicating more than one transcript is compatible. The disclosed quantification-in-memory algorithm was validated with existing software programs in (N. L. Bray et al., 2016, Nature biotechnology, vol. 34, no. 5, pp. 525-527), (R. Patro et al., 2017, Nature methods, vol. 14, no. 4), showing the same computation results and similar computing complexity, while the disclosed algorithm is optimized for PIM acceleration.

Although in the depicted example the k-mer length is 3, it is understood that any suitable k-mer length may be used, including but not limited to 2, 3, 4, 5, 6, 7, 8, 10, 12, 16, 24, 32, or 64.

To summarize, for PIM hardware implementation, the main operations of the disclosed quantification-in-memory are k-mer matching (based on XNOR) and AND logic for matched k-comps. For k-mer matching, one of the XNOR based match operands is fixed (i.e. pre-computed k-mers in the index-table), and the other operand is a fragment of input short read (i.e. a variable). This XNOR operation naturally matches with non-volatile PIM platform due to its greatly reduced leakage, non-volatility and parallel logic computation. Moreover, the matching operation among different index-tables is independent, where each computational array could be used as one matching engine to fully leverage the parallelism of the PIM architecture. For the AND operation, because it obeys the associative law, the whole bit-wise AND operation is divided into consecutive AND2 logic operations. Therefore, for each input fragment, after XNOR matching to identify the k-mer in each index-table, the corresponding k-comp will be activated to conduct AND logic with the previous AND output, updating the final output. The above analysis clearly shows that fast and parallel XNOR/AND logic operations are essential for PIM acceleration of quantification.

### PIM-Quantifier Architecture and Circuit

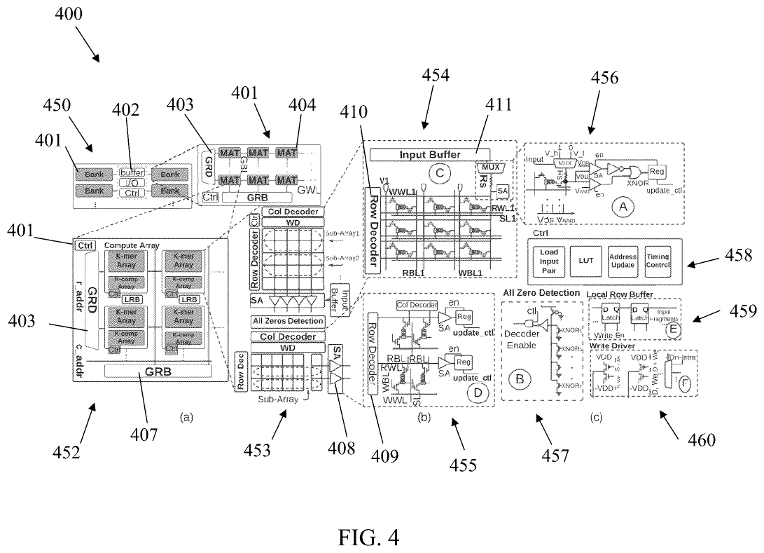

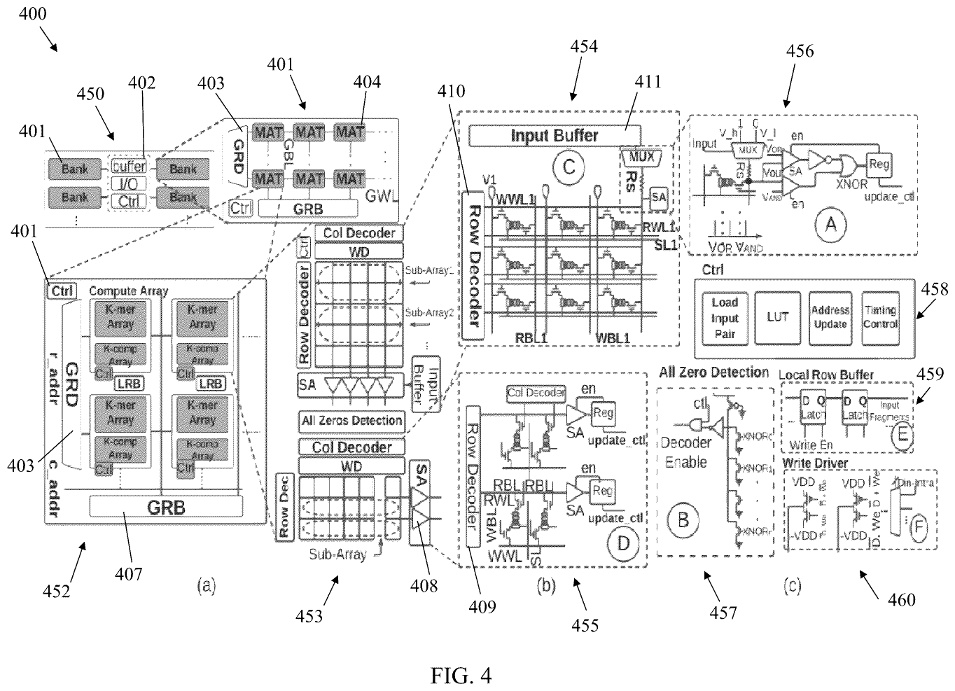

The disclosed PIM-Quantifier is designed to be an independent high-performance, parallel, and energy-efficient accelerator based on a conventional memory architecture. The hierarchy structure is given in FIG. 4. The main memory is composed of a set of MRAM chips 450. Each chip 450 contains multiple banks 401, sharing I/O, buffer 402, and control units. Each bank 401 is divided to multiple MATs 404 connected to a Global Row Decoder (GRD) 403 and a shared Global Row Buffer (GRB) 407. Each matrix (MAT) 404 comprises 2D arrays of computational Spin-Orbit Torque Magnetic Random Access Memory (SOT-MRAM) arrays 453 as demonstrated in FIG. 4. Each compute array includes two crucial sub-arrays, referred to herein as K-mer arrays 454 and K-comp arrays 455. The arrays may in some embodiments be configured to work in two modes (i.e. memory and in-memory computing mode) to process the computationally-intensive bit-wise XNOR and AND logic, respectively, required by the quantification-in-memory algorithm.

These two arrays store different types of data, but use the same designs of memory row/column decoder, Sense Amplifier (SA) 456, write driver 460, and local row buffers 459. The k-mer array architecture 454 is shown with a sample 3×3 array. Each SOT-MRAM cell is associated with the Write Word Line (WWL), Read Word Line (RWL), Write Bit Line (WBL), Read Bit Line (RBL), and Source Line (SL) to perform typical memory and in-memory computing operations. To program free-layer magnetization direction (thus low or high resistance level representing data ‘0’ and ‘1’) of SOTMRAM, flow of charge current (±y) through Spin Hall Metal (SHM) (Tungsten, β-W (C.-F. Pai et al., 2012, Applied Physics Letters, vol. 101, no. 12)) will cause accumulation of opposite directed electron spin on both surfaces of SHM due to spin Hall effect (X. Fong et al., 2016, IEEE TCAD, vol. 35, no. 1, pp. 1-22). Thus, a spin current flowing in ±z is generated and further produces spin-orbit torque (SOT) on the adjacent free magnetic layer, causing switch of magnetization, as well as the resistance of SOT-MRAM cell (i.e. writing data).

To perform memory read and PIM logic operations, the disclosed design adds a 2:1 MUX and a reference resistor (Rₛ) to each RBL, as shown in 456. For the typical memory read (e.g. M1), a read voltage is applied through the MUX's first input (V₁) to RBL1 and the sense current Iₛₑₙₛₑ flows from the selected SOT-MRAM cell's resistance (R_{M1}) to ground. Then, assuming R_{M1} and Rₛ as two elements of a voltage divider, the disclosed voltage-based sensing mechanism generates

$${V}_{s e n s e} ≃ \frac{{R}_{M 1}}{{R}_{M 1} + {R}_{S}} * {V}_{h / l}$$

at the input of the sense amplifier. This voltage is then compared with the memory mode reference voltage (V_{sense,P}<V_{ref}<V_{sense,AP}). Now, if the Vₛₑₙₛₑ is higher (/lower) than V_{ref}, i.e. R_{AP} (/R_{P}), then the output of the SA produces High (/Low) voltage indicating logic ‘1’ (/‘0’). In computing mode, the first operand is stored in the memory as a resistance state where the second operand (‘0’/‘1’) could be fed into the 2:1 MUX and selected by the ctrl unit. This effectively converts the binary input into a proportional sense voltage (Vₗ/Vₕ) to drive the RBL. In this way, the voltage-based sensing mechanism generates the corresponding Vₛₑₙₛₑ to various input combinations. Through selecting different reference voltages (En_{AND}, En_{OR}), the SA executes basic Boolean logic functions (i.e. AND and OR). For AND operations, V_{ref} is set at the midpoint of V_{AP}//V_{P} (‘1’, ‘0’) and V_{AP}//V_{AP} (‘1’, ‘1’). In the k-mer array, by activating two enables (En_{AND}, En_{OR}) simultaneously for all the RBLs, bulk bit-wise XNOR2 could be implemented in a single memory cycle quite efficiently. 455 represents the k-comp array developed to handle the consecutive AND operation of the selected k-comp, leveraging the same logic-in-memory design. The all-zero detection circuit in 457, as explained in the algorithm section, is used to detect whether the XNOR output is all zero (indicating that the current short read should be discarded). 457 is the shift register to generate fragments from the input short read.

### Mapping to PIM-Quantifier

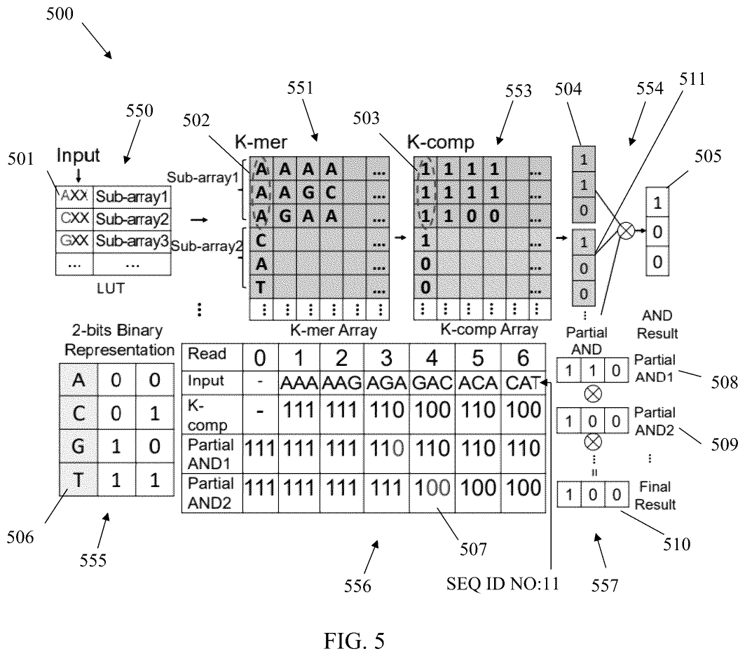

A method of deploying the mRNA quantification to PIM-Quantifier is disclosed in this section. To start, each pre-computed index-table is stored in the compute array comprising a k-mer array and a k-comp array. Both k-mers and k-comps are stored along bit-lines required by the property of the above discussed in-memory-logic designs and friendly for parallel computing. However, the k-mer table size could be very large, making it difficult to fit into one memory sub-array. Thus, introduced is an index-table partition method with the property that k-mers within the same memory sub-array share the same one or more front-end nucleotides (nt) depending on the total data size and memory sub-array size. The advantage of such partition method is that it could save several XNOR cycles for the front-end nt(s). For example, in the embodiment shown in FIG. 5, the k-mers in sub-array 502 all start with nt-‘A’. In some embodiments, for example where k-mers starting with different nt could be all stored in the same memory sub-array, such partitioning may not be necessary. When the input fragment is received, the first step in some embodiments is to locate which memory sub-array should be directed for the next matching stage, which could be implemented by a small look up table (LUT) 550.

In FIG. 5, assuming the input fragment is ‘AAA’, according to the LUT 550, the k-mers staring with ‘A’ are all stored in the sub-array-1 (502), thus activating the sub-array-1 502 for the XNOR based matching operation as discussed in the previous section. The matching operation in turn identifies one match, indicating ‘AAA’ is stored in the first column in the sub-array-1 502. Thus, the corresponding k-comp value stored in the first column (503) is ‘111’, which will be activated to conduct AND logic with the previous partial AND result stored in a latch. The result of the AND operation is then stored in the latch to update the partial AND result. When all fragments of a short read are processed, all the partial AND results from each sub-array stored in their latches 508, 509 will be collected to conduct a final round of AND operations to generate the final output 510, indicating which transcript is compatible with the current input short read. The AND operation of 504 and 511 represent the final operation after all input fragments are searched in the array. FIG. 5 provides an example to process 6 input fragments 556.

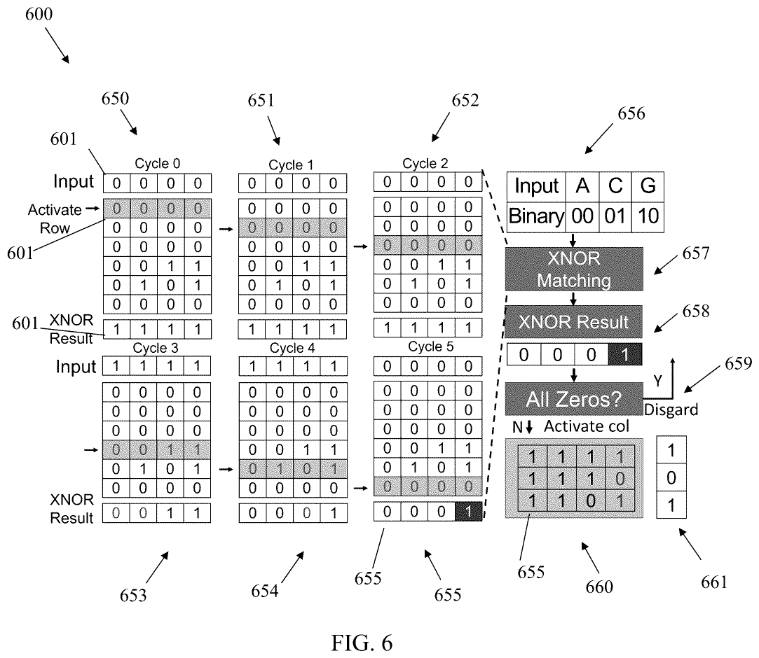

A detail view of the process is shown in FIG. 6. For XNOR-based matching within the sub-array, the first input fragment ‘ACG’ as one input example as shown in FIG. 6. Because there are only 4 types of nt, two bits are used to encode them defined in table 555 of FIG. 5. Thus, the input ‘ACG’ is encoded as ‘000110’. The system requires 6 cycles to perform XNOR based matching within the corresponding sub-array. As mentioned earlier, the k-mers are stored along bit-lines. The depicted 6×4 array includes 4 k-mers stored in the four columns, i.e. ‘AAA’, ‘AAG’, ‘ACA’, and ‘ACG’, from left to right. To maximize computing parallelism, multiple bit-lines (4 in this example) are activated at the same time to conduct parallel XNOR logic between the input and stored k-mers bit by bit. First, the system checks if all k-mers in this array begin with ‘A’. According to the XNOR result, it excludes those k-mers that are not staring with ‘A’, to narrow the search space for next nt. After two nt (i.e. 4 bits) matching (653), the XNOR based match result is ‘0011’, indicating the corresponding first two k-mers match the first two nt, i.e. ‘ACA’ and ‘ACG’. A similar XNOR based match then compares the input with the last nt, generating an XNOR based match result as ‘0001’ (655). This result indicates the last ‘1’ is matched with the input-‘ACG’. Then, the last k-comp in the corresponding k-comp array is activated for the following AND operation. Of course, it is possible there is no exact match in this k-mer array. In that case, the XNOR based match result should be all-zeros, which is detected by an all-zero detection circuit 659, one embodiment of which 457 is shown in FIG. 4. Correspondingly, no k-comp will be activated for the following AND operations.

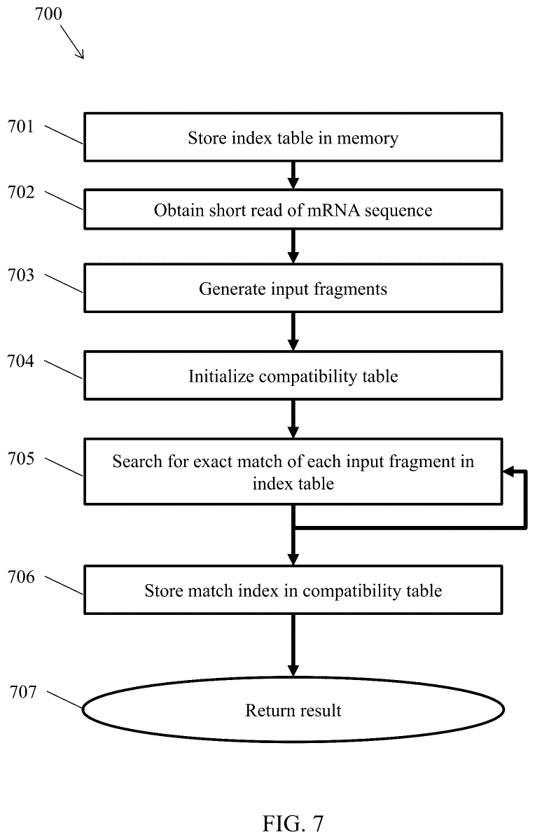

A method of calculating an abundance of an mRNA sequence within a gene is shown in FIG. 7. The method 700 includes the steps of storing an index table of the gene in a non-volatile memory in step 701, obtaining a short read of the mRNA sequence in step 702, generating a set of input fragments of size K from the mRNA sequence in step 703, initializing a compatibility table in the volatile memory in step 704, for each input fragment in the set of input fragments, searching for an exact match of the input fragment in the index table in step 705, if an exact match is found, storing a ‘1’ in a position in the compatibility table corresponding to the index of the exact match in step 706, and calculating an abundance of the mRNA sequence in the gene by calculating a final result from the compatibility table, and aggregating the transcripts compatible with the short read in step 707.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the system and method of the present invention. The following working examples therefore, specifically point out the exemplary embodiments of the present invention, and are not to be construed as limiting in any way the remainder of the disclosure.

### Performance Estimation: Experimental Setup

To assess the performance of PIM-Quantifier as the new PIM platform from circuit-level up to algorithm-level, a cross-layer comprehensive simulator was developed similar to (S. Angizi et al., 2019, 56^{th} DAC, pp. 1-6). The PIM-Quantifier's sub-array and peripheral circuits were designed in Cadence Virtuoso with the 45 nm NCSU Product Development Kit (PDK) library and then evaluated in Cadence Spectre for circuit-level performance parameters. The architecture-level simulator was based on NVSim where the configuration file is flexible and corresponds to a different array design and working mechanism. Thus, different types of PIM platforms can share a similar organization and simulator for fair comparison. For Content Addressable Memory (CAM) based designs, Nvsim-CAM (S. L. et al., 2016, ICCAD, pp. 1-7) was used to estimate their performances. In addition to the architecture simulator, MATLAB was used to pre-process the real genome data. The cross-layer simulator could evaluate latency, energy, and throughput for the alignment-free based quantification with the human genome hg38 dataset.

A process including 1 million short reads was used with a length of 101 as test inputs. A total of 22000 genes (index-tables) were tested during the process. Each index-table contains 3000 to 10000 k-mers with length of 25. The PIM-Quantifier's memory array was configured with 256 rows and 1024 columns, 8×2 MATs (with 1/1 as row/column activation) per bank organized in H-tree routing manner, 64×64 banks (with 1/1 as row/column activation) in each memory group. In most use cases, 65K sub-arrays are sufficient. In the rest of this section, the bulk bitwise operations were analyzed for the proposed platform. The Monte-Carlo simulation was also performed to show its stability. Then, more detailed experiments were conducted to compare different PIM hardware platforms, to perform data-mapping optimization, and to include real gene data.

### Performance Estimation: Circuit Level Analysis

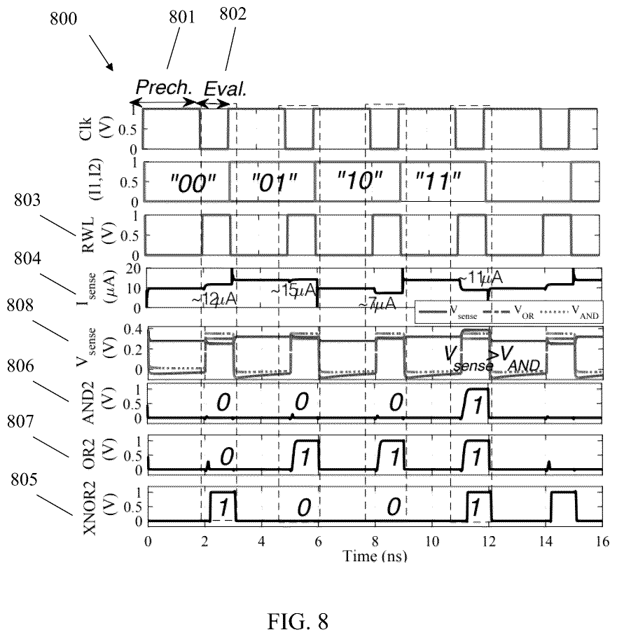

FIG. 8 depicts the transient simulation results of a single k-mer/k-comp sub-array based on the architecture shown in FIG. 4. For the sake of clarity, it was assumed that a 3 ns period clock synchronizes the write and read operations. However, it is understood that any suitable clock period could be used for a reliable read operation, including but not limited to a 5 ns, 4 ns, 2 ns, 1 ns, or 500 ps clock. During the precharge phase 801 of the SA (Clk=1), the Vwrite voltage was set and applied to the WBL to change the selected SOT-MRAM cell's resistance to R_{low}=5.9 kΩ or R_{high}=15.7 kΩ. This way, the first operand is stored into the memory bit-cell as a resistance state. Prior to the evaluation phase (Eval.) 802 of the SA, WWL and WBL were grounded. The second operand (‘0’/‘1’) was converted to a sense voltage (400 mV/500 mV) and fed to the RBL. In the evaluation phase, RWL 803 goes high. Depending on the resistance state of the SOT-MRAM bit-cell, Vₛₑₙₛₑ was generated through the resistive voltage divider with the Rs=5 kΩ as the first input of the SA, when V_{ref} was applied at the second input of the SA. The comparison between Vₛₑₙₛₑ and V_{ref} for all possible input cases are plotted in FIG. 8. It was observed when Vₛₑₙₛₑ<V_{OR} (only in the first evaluation phase 802), the SA output a binary ‘0’, whereas the system output 805 was “1” Shown are two SA outputs, one with the input V_{and}, the output represented by AND2 806 and another SA with the input Vₒᵣ, represented by Or2 807. The Iₛₑₙₛₑ 804 was plotted to analyze possible read disturbance when applying the Vₛₑₙₛₑ. It was observed that in the worst case Iₛₑₙₛₑ was 15 μA and I_{write} was 130 μA. The Vₛₑₙₛₑ 808 compares with both Vₒᵣ 807 and V_{and} 806 simultaneously by two SA outputs.

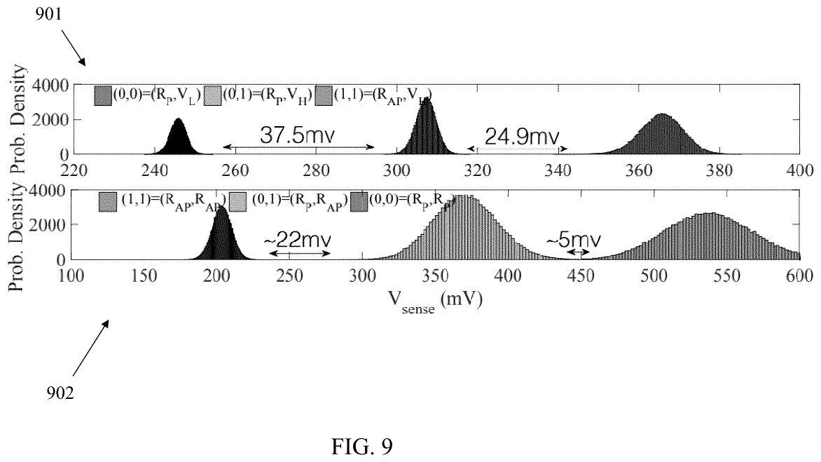

To validate the variation tolerance of the sensing circuit, a worst-case scenario Monte-Carlo simulation was performed with 100000 trials. A σ=5% variation was added to the Resistance-Area product (RA_{P}), and a σ=10% process variation (typical MTJ conductance variation (X. Fong et al., 2016, Proceedings of the IEEE, vol. 104, no. 7)) was added on the TMR. The simulation result of sense voltage (Vₛₑₙₛₑ) distributions for the presented one-row activation in-memory mechanism is shown in graph 901 of FIG. 9. It was observed that a 34.2 mv and 18.7 mv sensing margin were achieved between three possible cases. Graph 902 of FIG. 9 shows the sensing margin for the conventional 2-row activation PIM logic. It was observed that the presented design provides larger sensing margins especially when it comes to “01” and “11” margin. This was mainly due to the fact that, assuming R_{M1} and R_{M2} as two MRAM cells located in a same bit-line and Rₛ as the reference resistor, the voltage-based sensing mechanism provides

$${V}_{s e n s e} ≃ \frac{{R}_{M 1}}{{R}_{M 1} + {R}_{S}} * {V}_{h / l} ,$$

where the current-based two-row activation mechanism (S. Angizi et al., 2018, 23rd ASP-DAC, pp. 111-116) provides Vₛₑₙₛₑ≈Iₛₑₙₛₑ*(R_{M1}//R_{M2}), because the parallel resistance was virtually half of the resistance of a single cell.

### Performance Estimation: Experimental Results

Because there is no prior PIM based hardware acceleration of mRNA quantification, to conduct a fair comparison, several representative non-volatile PIM designs were reimplemented from CAM ((Li-Yue Huang et al., 2014, Symposium on VLSI Circuits Digest of Technical Papers, pp. 1-2); (J. Li et al., 2014, IEEE Journal of SolidState Circuits, vol. 49, no. 4, pp. 896-907); J. Li et al., 2014, IEEE Journal of SolidState Circuits, vol. 49, no. 4, pp. 896-907), IMCE (S. Angizi et al., 2018, 23rd ASP-DAC, pp. 111-116), Pinatubo (S. Li et al., 2016, in 53rd DAC, pp. 1-6), to also deploy the quantification-in-memory algorithm in those platforms. Similar to the proposed design, the CAM based platforms were configured to only store one XNOR operand in the memory array and convert the other operand as a voltage/current input. Thus, those CAM based platforms share a memory structure similar to the proposed design. The IMCE and Pinatubo needed to write both XNOR operands into the non-volatile memory array for logic functions, thus larger memory array sizes were needed for these two platforms to use the same index table partition. The MAT and bank organization remained the same as other platforms. The results were also compared to results measured from CPU (Intel E5-2620) performance using state-of-the-art mRNA transcript quantification software-Kallisto (N. L. Bray et al., 2016, Nature biotechnology, vol. 34, no. 5, pp. 525-527).

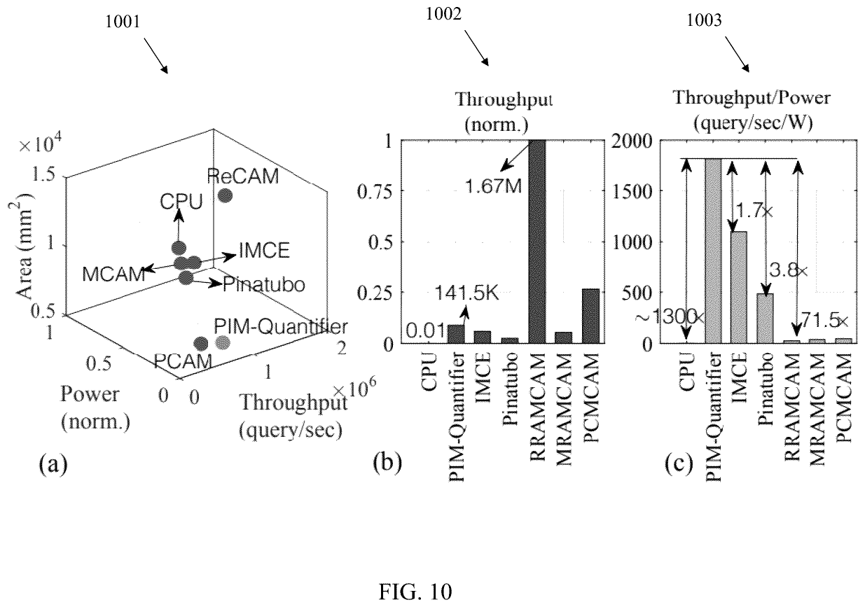

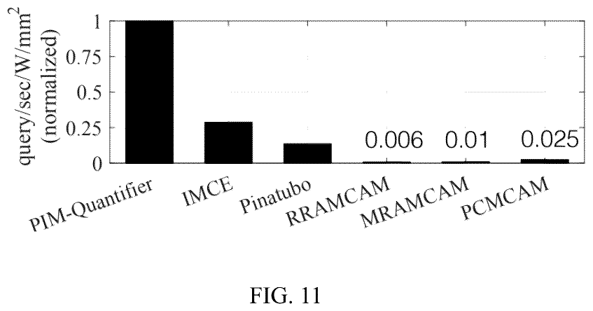

Table 1 below, FIG. 10 and FIG. 11 summarizes the key performance differences between different PIM platforms and CPUs. As a result of the proposed optimized circuit designs, the PIM-Quantifier requires less read & write energy than the IMCE or Pinatubo. Unlike that of IMCE and Pinatubo, the input of PIM-Quantifier does not need to be written into a memory array for computing, eliminating extra input operand writing power/latency and reducing the amount of memory required. As expected, CAM based platforms achieve the highest throughput due to the high parallel matching scheme. However, such high throughput comes at the cost of extremely high power consumption. Moreover, those platforms need a 4-transistor and 2-resistor (4T2R) cell structure, making each memory cell two times larger than other PIM platforms. If defining the efficiency as ‘throughput/power’, as shown in graph 1003 of FIG. 10, PIM-Quantifier is 1.7×-71.5× more efficient (in terms of throughput/power) than other PIM based platforms. The CPU implementation has the worst efficiency in this case, almost three orders of magnitude worse than all PIM based platforms. FIG. 11 shows the normalized comparison of throughput/power/area of different PIM platforms, showing PIM-Quantifier greatly outperforms others.

TABLE 1

| | PIM- | | IMCE | | Pinatubo | | RRAM | | MRAM | | PCM | |
|---|---|---|---|---|---|---|---|---|---|---|---|---|
| | Quantifier | | 512 × 1024 | | 512 × 1024 | | CAM | | CAM | | CAM | |
| Technology(nm) | 45 | nm | 45 | nm | 45 | nm | 45 | nm | 45 | nm | 45 | nm |
| Latency(ns) | 3.69 | ns(read) | 3.691 | ns | 6.994 | ns | 7.79 | ns | 150.61 | ns | 30.69 | ns |
| | 1.66 | ns(write) | 1.840 | ns | 5.968 | ns | 17.76 | ns | 32.59 | ns | 100 | ns |
| Energy(Read/row) | 90.94 | pJ | 135.940 | pJ | 137.436 | pJ | 54.43 | pJ | 697.28 | pJ | 116.7 | pJ |
| Energy(Write/row) | 61.34 | pJ | 92.092 | pJ | 1.088 | nJ | 1.200 | nJ | 147.96 | pJ | 7.34 | nJ |

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of calculating an abundance of an mRNA sequence within a gene, comprising:
storing an index table of the gene in a non-volatile memory, the index comprising a set of nucleotide substrings of length K and having a size in bits of at least 2K;
obtaining a short read of the mRNA sequence comprising N nucleotides;
generating a set of input fragments of size K from the mRNA sequence using a sliding window;
initializing a compatibility table in a volatile memory corresponding to a set of T transcripts of the gene;
for each input fragment in the set of input fragments, searching for an exact match of the input fragment in the index table;
if an exact match is found, storing a ‘1’ in a position in the compatibility table corresponding to the index of the exact match;
calculating a final result having a length T from the compatibility table, wherein each of the T positions of the final result corresponds to one of the set of T transcripts of the gene, and wherein a 1 in the position indicates that the transcript is compatible with the short read; and
calculating an abundance of the mRNA sequence in the gene by aggregating the transcripts compatible with the short read;
wherein the calculating step is performed on the same integrated circuit as the non-volatile memory.

2. The method of claim 1, wherein the exact match of the input fragment to the index table is calculated with a bitwise XNOR.

3. The method of claim 2, wherein the bitwise XNOR is performed in a single clock cycle.

4. The method of claim 1, wherein the step of calculating the final result comprises the step of performing a bitwise AND operation between a first set of bits in the compatibility table and a second set of bits in the compatibility table, and storing the result in the compatibility table.

5. The method of claim 1, further comprising detecting whether all the bits in a subset of the compatibility table are set to 0.

6. The method of claim 1, wherein the input fragments are generated using at least one shift register.

7. The method of claim 1, further comprising storing the index table and the compatibility table in the same bank.

8. The method of claim 1, further comprising the step of splitting the index table into multiple index sub-tables stored in different areas of the memory.

9. The method of claim 8, wherein the index table is split based on the first nucleotide in the input fragments.

10. The method of claim 8, further comprising recording an index of the multiple index sub-tables in a look-up table.

11. The method of claim 10, further comprising querying the look-up table for the correct index sub-table before searching for an exact match of the input fragment in the index table.

12. A system for in-memory calculation of an abundance of an mRNA sequence within a gene, comprising:
a non-volatile computer-readable memory storing a set of binary values, the non-volatile computer-readable memory comprising a plurality of read bitlines and read wordlines;
a computational array communicatively connected to the non-volatile computer-readable memory, comprising:
an input shift register configured to generate binary substrings from an input binary string;
a multiplexer having at least two inputs, having at least one output electrically connected via a resistor to at least one read bitline, configured to selectively change the voltage of the read bitline at the input to a sense amplifier during a read operation; and
a set of combinatorial logic gates electrically connected to an output of the sense amplifier, configured to return a result and store the result in the non-volatile computer-readable memory; and
a processor configured to calculate the abundance of an mRNA sequence within a gene by storing an index table of the gene in the non-volatile memory, generating a set of input fragments from the mRNA sequence, searching for exact matches of the input fragments in the index table using the multiplexer and the set of combinatorial logic gates to calculate a set of transcripts compatible with the short read, and calculating the abundance of the mRNA sequence in the gene by aggregating the transcripts compatible with the input binary string.

13. The system of claim 12, wherein the set of combinatorial logic gates comprises at least one XNOR gate.

14. The system of claim 13, wherein the set of combinatorial logic gates comprises an XNOR gate with one inverted input.

15. The system of claim 12, wherein the multiplexer has exactly two inputs and one output.

16. The system of claim 12, wherein the computational array comprises first and second sense amplifiers configured to have different threshold voltages.

17. The system of claim 12, further comprising an all-zero detection unit configured to detect when a calculated result vector contains all zeros.

18. The system of claim 12, wherein the computational array and the volatile computer-readable memory are positioned in a single integrated circuit.

19. The system of claim 18, wherein the processor is positioned in the single integrated circuit.

20. The system of claim 12, wherein the processor is further configured to divide the index table into multiple index sub-tables stored in different areas of the memory.


SEQUENCE LISTING (11 sequences)

SEQ ID NO: 1 - RNA, 10 nt, synthetic construct
  source 1..10: mol_type = other RNA
actatgagct

SEQ ID NO: 2 - RNA, 11 nt, synthetic construct
  source 1..11: mol_type = other RNA
gcctagtagct

SEQ ID NO: 3 - RNA, 10 nt, synthetic construct
  source 1..10: mol_type = other RNA
ctacacacat

SEQ ID NO: 4 - RNA, 11 nt, synthetic construct
  source 1..11: mol_type = other RNA
gctatcgagat

SEQ ID NO: 5 - RNA, 11 nt, synthetic construct
  source 1..11: mol_type = other RNA
agtagctttcg

SEQ ID NO: 6 - RNA, 12 nt, synthetic construct
  source 1..12: mol_type = other RNA
aaatatactctc

SEQ ID NO: 7 - RNA, 18 nt, synthetic construct
  source 1..18: mol_type = other RNA
aagtgaaaatatactctc

SEQ ID NO: 8 - RNA, 18 nt, synthetic construct
  source 1..18: mol_type = other RNA
cactataaatatactctc

SEQ ID NO: 9 - RNA, 15 nt, synthetic construct
  source 1..15: mol_type = other RNA
aagagagtatttcac

SEQ ID NO: 10 - RNA, 15 nt, synthetic construct
  source 1..15: mol_type = other RNA
aaaactaaatatact

SEQ ID NO: 11 - RNA, 18 nt, synthetic construct
  source 1..18: mol_type = other RNA
aaaaagagagacacacat